(19) United States
(12) Patent Application Publication
(10) Pub. No.: US 2026/0237488 A1
(43) Pub. Date: Aug. 13, 2026

(54) AUTOMATED SEGMENTATION OF CONTRAST ENHANCED ULTRASOUND IMAGES

(71) Applicant: REGENTS OF THE UNIVERSITY OF MICHIGAN, Ann Arbor, MI (US)

(72) Inventors: Kourosh Kalayeh, Ann Arbor, MI (US); J. Brian Fowlkes, Ann Arbor, MI (US); Bryan Sack, Ann Arbor, MI (US); William W. Schultz, Ann Arbor, MI (US); John O. Delancey, Ann Arbor, MI (US)

(21) Appl. No.: 19/447,269

(22) Filed: Jan. 13, 2026

Related U.S. Application Data
(60) Provisional application No. 63/756,435, filed on Feb. 10, 2025.

(51) Int. Cl. G16H 30/40; G06T 7/10; G06V 10/25

(52) U.S. Cl. CPC G16H 30/40; G06T 7/10; G06V 10/25; G06T 2207/10132

(57) ABSTRACT

A method for performing automated segmentation of contrast enhanced ultrasound images includes obtaining, by an ultrasound sensor, a first ultrasound image of a body region of a patient. The method includes identifying an area of interest in the first ultrasound image and performing first stage image processing on the first ultrasound image. The method includes determining a closed perimeter in the first ultrasound image from the first stage image processing that is indicative of a region of interest and includes a portion of the area of interest. The method further includes performing second stage image processing on the first ultrasound image, including limiting at least part of the second stage image processing to the region of interest indicated by the closed perimeter, and determining a measurement region.

### CROSS-REFERENCE TO RELATED APPLICATION

This application claims the benefits of priority to United States Provisional Patent Application No. 63/756,435, filed Feb. 10, 2025, titled AUTOMATED SEGMENTATION OF CONTRAST ENHANCED ULTRASOUND IMAGES, the contents of which are hereby expressly incorporated into the present application by reference in their entirety.

### STATEMENT OF GOVERNMENT SUPPORT

This invention was made with government support under DK122379 awarded by the National Institutes of Health. The government has certain rights in the invention.

### FIELD OF THE DISCLOSURE

The invention generally relates to methods and systems for improved ultrasound measurements, and more particularly, for automated segmentation of contrast enhanced ultrasound images. The methods and systems may be used for ultrasound-based estimations of an ambient pressure, such as urinary tract pressure.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Urologists and gynecologists rely on urodynamic tests to evaluate bladder function. A key component of these tests is the cystometrogram (CMG), which measures bladder pressure. During a CMG test a urethral catheter is inserted into the bladder and left there. The bladder is then filled with saline and pressure measured during filling and voiding. The patient is asked to report different bladder sensation (first desire to void, strong desire to void, etc.). The major challenge of this test is the presence of the indwelling urethral catheter results in discomfort for the patient such that the patient sometimes finds it challenging to distinguish the sensation of having a catheter in their urethra from other bladder sensations. In addition, during micturition, non-physiological voiding conditions occur from the presence of the catheter, and partial obstruction to flow can occur causing falsely elevated pressures and reduced flow rates.

### SUMMARY OF THE INVENTION

Techniques and systems are provided for performing a pressure measurement of fluid in the urinary tract. In a specific implementation, a method for performing automated segmentation of contrast enhanced ultrasound images across the urinary tract is disclosed. The method includes obtaining, by an ultrasound sensor, a first ultrasound image of a body region of a patient, the body region including one or more tissues or organs of the urinary tract. A processor identifies an area of interest in the first ultrasound image and further performs first stage image processing on the first ultrasound image. The method further includes determining a closed perimeter in the first ultrasound image from the first stage image processing with the closed perimeter indicative of a region of interest of the first ultrasound image that includes a portion of the area of interest. The method then includes performing second stage image processing on the first ultrasound image, including limiting at least part of the second stage image processing to the region of interest indicated by the closed perimeter. The processor then determines a measurement region in the first ultrasound image from the second stage image processing and the region of interest indicated by the closed perimeter.

In examples of the current implementation, the method further includes determining, by the processor, an ultrasound contrast signal intensity in the measurement region of the first ultrasound image. In additional examples, determining the ultrasound contrast signal intensity may comprise identifying one or more subharmonic signal intensities.

In examples of the current implementation, the method further includes determining, by the processor, an ambient pressure in the measurement region from the ultrasound contrast signal intensity.

In additional examples, the first stage image processing includes one or more of an image blur, semi-horizontal tissue layer analysis, an Otsu thresholding technique, a binary thresholding, application of a mask, an erosion, a smoothing function, a distance transform, a contour function, and a spline. In more examples, the second stage image processing includes one or more of an image blur, semi-horizontal tissue layer analysis, an Otsu thresholding technique, a binary thresholding, application of a mask, an erosion, a smoothing function, a distance transform, a contour function, and a spline.

In additional examples, the second stage image processing includes one or more of noise reduction, horizontal tissue layer analysis, adaptive thresholding, binarization, mask application, an erosion, a dilation, a smoothing function for shape refinement, a distance transformation for region analysis, small blob removal, convex hull extraction for contour detection , and contour smoothing. In more examples, the second stage image processing includes one or more of noise reduction, horizontal tissue layer analysis, adaptive thresholding, binarization, mask application, an erosion, a dilation, a smoothing function for shape refinement, a distance transformation for region analysis, small blob removal, convex hull extraction for contour detection , and contour smoothing

In examples, determining the ambient pressure from the ultrasound contrast signal intensity includes determining a distribution of ultrasound signal within the measurement region, and correlating ambient pressure in the measurement region with the distribution of ultrasound signal. In the current example, determining the distribution of ultrasound signal may include generating a histogram of ultrasound signal in the measurement region.

In another implementation, disclosed is a system for automated segmentation of contrast enhanced ultrasound images. The system includes an ultrasound sensor, a processor configured to execute machine readable instructions, and a non-transitory computer- readable memory having machine readable instructions stored thereon. When the processor executes the machine-readable instructions, the system obtains, by the ultrasound sensor, a first ultrasound image of a body region of a patient, the body region including one or more tissues or organs of the urinary tract, identifies an area of interest in the first ultrasound image, performs first stage image processing on the first ultrasound image and determines a closed perimeter in the first ultrasound image from the first stage image processing, the closed perimeter indicative of a region of interest of the first ultrasound image that includes a portion of the area of interest , performs second stage image processing on the first ultrasound image, including limiting at least part of the second stage image processing to the region of interest indicated by the closed perimeter, and determines a measurement region in the first ultrasound image from the second stage image processing and the region of interest indicated by the closed perimeter.

In examples, the machine-readable instructions, when executed by the processor, cause the system to determine an ultrasound contrast signal intensity in the measurement region of the first ultrasound image. In examples, determining the ultrasound contrast signal intensity comprises identifying one or more subharmonic signal intensities.

In examples, the machine-readable instructions, when executed by the processor, cause the system to determine an ambient pressure in the measurement region from the ultrasound contrast signal intensity.

In additional examples of the system, the first stage image processing includes one or more of noise reduction, horizontal tissue layer analysis, adaptive thresholding, binarization, mask application, an erosion, a dilation, a smoothing function for shape refinement, a distance transformation for region analysis, small blob removal, convex hull extraction for contour detection , and contour smoothing. In more examples, the second stage image processing includes one or more of noise reduction, horizontal tissue layer analysis, adaptive thresholding, binarization, mask application, an erosion, a dilation, a smoothing function for shape refinement, a distance transformation for region analysis, small blob removal, convex hull extraction for contour detection , and contour smoothing.

In further examples, to determine the ambient pressure from the ultrasound contrast signal intensity, the machine-readable instructions, when executed by a processor, further cause the system to determine a distribution of ultrasound signal within the measurement region and correlate ambient pressure in the measurement region with the distribution of ultrasound signal. Further, to determine the distribution of ultrasound signal, the machine-readable instructions, when executed by a processor, may cause the system to generate a histogram of ultrasound signal in the measurement region.

In yet another implementation, disclosed is one or more non-transitory computer-readable media. The non-transitory computer-readable media stores computer executable instructions that, when executed via one or more processors, cause one or more systems to: obtain a first ultrasound image of a body region of a patient, the body region including one or more tissues or organs of the urinary tract; identify an area of interest in the first ultrasound image, perform first stage image processing on the first ultrasound image and determine a closed perimeter in the first ultrasound image from the first stage image processing, the closed perimeter indicative of a region of interest of the first ultrasound image that includes a portion of the area of interest , perform second stage image processing on the first ultrasound image, including limiting at least part of the second stage image processing to the region of interest indicated by the closed perimeter, and determine a measurement region in the first ultrasound image from the second stage image processing and the region of interest indicated by the closed perimeter.

In examples, the computer executable instructions, when executed via one or more processors, cause one or more systems to determine an ultrasound contrast signal intensity in the measurement region of the first ultrasound image. In examples, the computer executable instructions, when executed via one or more processors, cause one or more systems to determine an ambient pressure in the measurement region from the ultrasound contrast signal intensity.

In examples, to determine the ultrasound contrast signal intensity, the computer executable instructions further cause the one or more systems to: identify one or more subharmonic signal intensities; determine a distribution of the one or more subharmonic signal intensities within the measurement region; and correlate ambient pressure in the measurement region with the distribution of subharmonic signal intensities.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 is a schematic illustration of an example scenario for performing ultrasound imaging using an ultrasound probe and determining pressure measurements within tissues and organs, in accordance with an example.

FIG. 2 is a block diagram of an example system for performing the ultrasound imaging and pressure measurement methods, in accordance with an example.

FIG. 3 is an example ultrasound image of a bladder of a patient taken with an ultrasound probe, such as that shown in FIG. 1, and the system of FIG. 2, in accordance with an example.

FIG. 4 is a flow diagram of a method for performing automatic segmentation of contrast-enhanced ultrasound image and calculating pressure measurements in tissues and organs, such as in the bladder, in accordance with an example.

FIG. 5A is an example ultrasound image with a blur function applied as part of first stage image processing, in accordance with an example.

FIG. 5B is an example mask layer determined from semi-horizontal tissues of the ultrasound image of FIG. 5B, as part of first stage image processing, in accordance with an example.

FIG. 5C is an example ultrasound image with pixels removed according to the mask of FIG. 5B as part of first stage processing, in accordance with an example.

FIG. 5D is an example image of pixels generated from the ultrasound image of FIG. 5C with an Otsu thresholding applied as part of first stage image processing, in accordance with an example.

FIG. 5E is an example image of pixels with the semi-horizontal tissue layer found in FIG. 5B as part of first stage image processing, in accordance with an example.

FIG. 5F is an example image of pixels with a morphological operation (here, an erosion filter) applied as part of first stage image processing, in accordance with an example.

FIG. 5G is an example image of pixels with a smoothing function applied as part of first stage image processing, in accordance with an example.

FIG. 5H is an example image of pixels with a distance transform applied as part of first stage image processing, in accordance with an example.

FIG. 5I is an example image of pixels with a binary thresholding applied as part of first stage image processing, in accordance with an example.

FIG. 5J is an example image of pixels after the removal of small pixel groupings applied as part of first stage image processing, in accordance with an example.

FIG. 5K is an example ultrasound image with a convex hull determined from the foreground pixels of FIG. 5J, in accordance with an example.

FIG. 5L is an example ultrasound image after applying a smoothing function to the convex hull of FIG. 5K as part of first stage image processing, in accordance with an example.

FIG. 6 is an ultrasound image with a closed perimeter as determined by first stage image processing, in accordance with an example.

FIG. 7A is an example ultrasound image with a blur function applied as part of second stage image processing, in accordance with an example.

FIG. 7B is an example image of pixels generated from the ultrasound image of FIG. 5C with an Otsu thresholding applied as part of second stage image processing, in accordance with an example.

FIG. 7C is an example image of pixels after applying a closed perimeter as part of second stage image processing, in accordance with an example.

FIG. 7D is an example image of pixels with an erosion filter applied as part of second stage image processing, in accordance with an example.

FIG. 7E is an example image of pixels with a smoothing function applied as part of second stage image processing, in accordance with an example.

FIG. 7F is an example image of pixels with a distance transform applied as part of second stage image processing, in accordance with an example.

FIG. 7G is an example image of pixels with a binary thresholding applied as part of second stage image processing, in accordance with an example.

FIG. 7H is an example image of pixels after the removal of small pixel groupings applied as part of second stage image processing, in accordance with an example.

FIG. 7I is an example ultrasound image with a convex hull determined from the foreground pixels of FIG. 7H, in accordance with an example.

FIG. 7J is an example ultrasound image after applying a smoothing function to the convex hull of FIG. 7I to identify a measurement region of the ultrasound image, in accordance with an example.

FIG. 8A provides a histogram of a pixel distribution of ultrasound contrast signal intensity for a measurement region of an ultrasound image, in accordance with an example.

FIG. 8B is a box plot of ultrasound intensity of a measurement region of an ultrasound image, in accordance with an example.

FIG. 9A illustrates an example of a correlation between a bladder pressure and a microbubble sub-harmonic signal amplitude.

FIG. 9B illustrates a linear relationship between a change in pressure and a change in subharmonic signal.

### DETAILED DESCRIPTION

Provided are techniques for measuring bladder pressure without use of a catheter, thereby avoiding the falsely elevated pressures and reduced flow rates associated with techniques requiring an indwelling urethral catheter. Specifically, the techniques and methods are ultrasound-based and use sub-harmonic-aided pressure estimation (SHAPE) to measure voiding bladder pressure catheter-free. The described methods may be used to improve the abilities of SHAPE technologies for performing pressure measurements. For example, current SHAPE techniques suffer from increased errors due to organ motion and movement of instruments, such as ultrasound imagers and probes, during imaging. The described methods and systems provide insight into pressures in the urinary tract and allow for enhanced understanding and evaluation of the urinary tract, including the bladder. The disclosed systems reduce the reliance on invasive pressure measurement sensors during bladder filling and devices which inherently introduce additional error into pressure readings, and also are often uncomfortable for patients resulting in patient induced errors in measurements as well. This discomfort from the measuring catheter can make it difficult for patients to separate discomfort due to the continued presence of an indwelling catheter from the bladder sensations they are asked to report relevant to sensations of bladder filling. Using an ultrasound probe, and automated machine vision and image processing provides a more systematic and standardized method for measuring pressure across the urinary tract. While the disclosed examples are largely directed to using the disclosed method of automatic segmentation of contrast-enhanced ultrasound images to provide pressure measurements of the bladder, it should be understood that the disclosed methods may be implemented in measuring other organs and body systems. The method of automatic segmentation has applications outside of pressure measurement and may facilitate ultrasound analysis in a variety of different clinical applications.

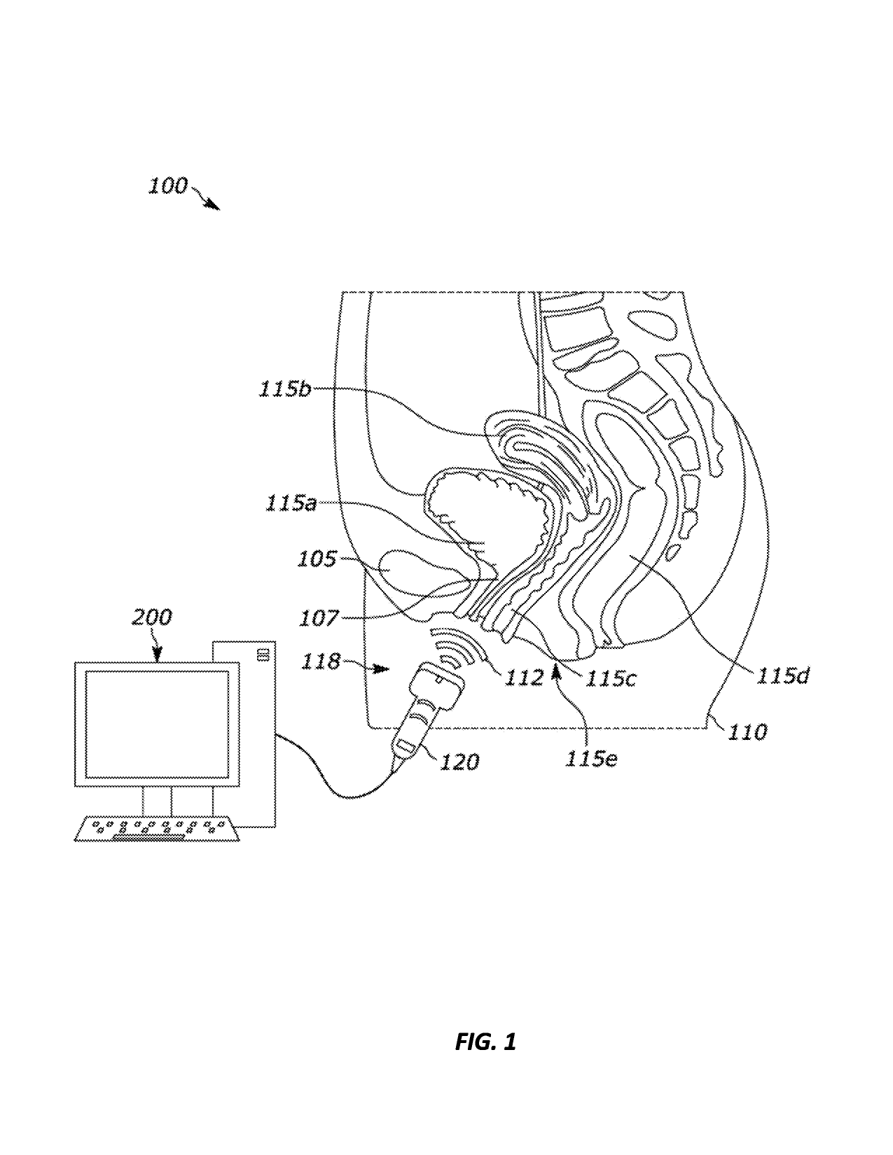

FIG. 1 is a schematic illustration of an example scenario 100 for performing ultrasound imaging and pressure measurements of the urinary tract, and specifically, of the bladder. In the example scenario 100 of FIG. 1, an ultrasound probe 120 is placed against the body 110 of a patient to provide ultrasound waves 112 to the body 110 and to detect reflected ultrasound vibrations during both the filling and voiding phases of bladder activity. A contrast agent, such as microbubbles, is administered prior to the collection of ultrasound waves 112 to enable sub-harmonic-aided pressure estimation (SHAPE). A gel, such as an ultrasound gel, may be applied to a portion of the body 110 and the ultrasound probe 120 may be placed against the portion of the body 110 having the ultrasound gel to increase the coupling of the ultrasound waves 112 into the body 110 of the patient. The ultrasound probe 120 may then receive reflected ultrasound vibrations to image tissues and organs inside of the body 110. In the illustrated example, the ultrasound probe 120 is positioned against the body 110 to image a pelvic region of the patient. The ultrasound probe may image one or more of a pubis 105, urethra 107, bladder 115a, uterus 115b, vagina 115c, rectum 115d, and perineal body 115e, among other tissues and/or organs. In particular, in the figures discussed below, the bladder 115a has been imaged.

As shown in FIG. 1, the ultrasound probe 120 is positioned on the patient for imaging. The ultrasound probe 120 may be held in a variety of positions on the patient. For example, the ultrasound probe may be positioned for either transabdominal imaging or transperineal imaging. Movement of the ultrasound probe 120 or organs during imaging causes the positions of organs to change in the resultant ultrasound images. This movement may introduce errors in diagnosis and measurements such as for determining pressure in tissues and organs. Therefore, the movement of the ultrasound probe 120 must be decoupled from the identification and isolation of organs and tissues in images for performing pressure measurements. To address this challenge, specific tissues and organs of interest are identified and isolated in independent images to determine pressure within these structures over time and in real-time.

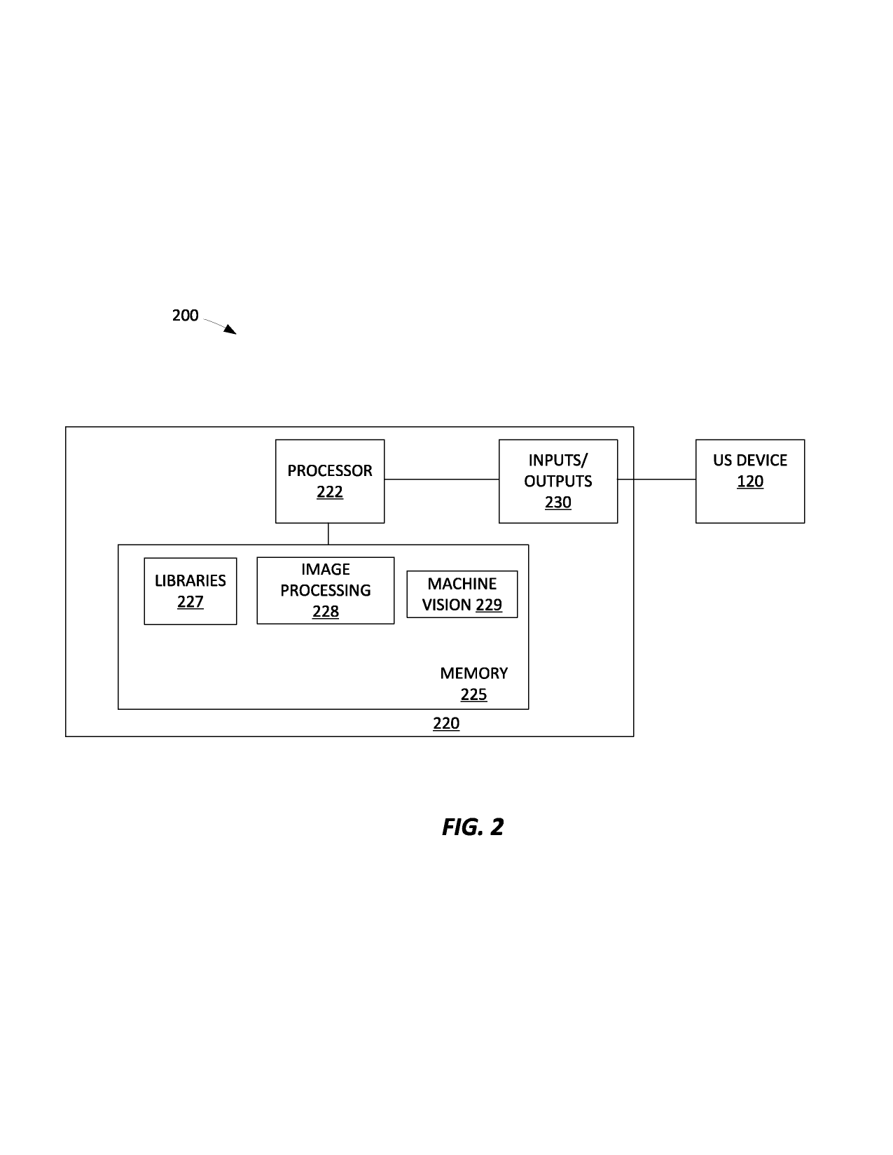

The ultrasound probe 120 generates a signal indicative of the detected reflected ultrasound waves and provides the signal to one or more systems 200 for performing image processing, machine vision operations, and for displaying one or more ultrasound images and videos. FIG. 2 is a block diagram of an example system 200 for performing the ultrasound image, automatic segmentation, and organ pressure measurement methods described herein. The system 200 may be used to perform any of the methods disclosed herein. The system 200 includes one or more processors 222 that execute machine readable instructions for performing organ pressure measurements according to the described methods. The processor may access one or more memories 225 to retrieve data, store data, and retrieve or store machine readable instructions. The one or more memories 225 may store one or more software or language libraries 227 (e.g. Python open source libraries, or other software and processing libraries), image processing algorithms 228, and machine vision processes 229. The image processing algorithms 228 may include noise reduction (e.g., blurring operations), horizontal tissue layer analysis (e.g., using gradient operators such as Sobel, and edge detection algorithms like Canny) , adaptive thresholding (e.g., Otsu), binarization (e.g., binary thresholding), mask application, morphological operations (e.g., erosion, dilation), smoothing functions for shape refinement, distance transformation for region analysis, small blob removal, convex hull extraction for contour detection, and contour smoothing, and others.

The system 200 further includes one or more input/output ports 230 or devices. For example, the input/output ports 230 may include wired or wireless communication channels that receive or provide data to external networks, servers, and devices. The input/output ports 230 may include display devices such as monitors and touchscreens to provide images and video to a user. The input/output ports 230 may further include input devices such as one or more keyboards, mice, touchscreens, etc. In the example provided, the probe 120 provides data indicative of ultrasound images and video to the system 200 vie the input/output ports 230.

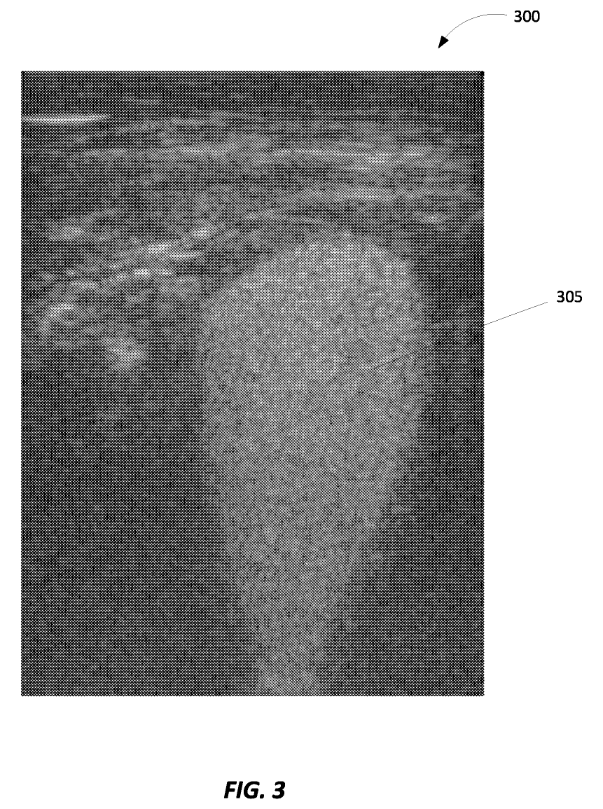

FIG. 3 is an example ultrasound image 300 of a bladder 305. The image includes the contrast agent infused into the bladder 305 and some abdominal tissue layers that the scanner failed to suppress. The example image 300 of FIG. 3 will be further used to illustrate the methods of machine vision and image processing for performing pressure measurements described herein.

The image 300 of FIG. 3 was obtained in contrast mode using a SHAPE technique. A curvilinear ultrasound transducer was used to send acoustic beams into the tissue and receive acoustic echoes. The ultrasound transducer sweeps the acoustic beam along multiple angular directions within a tissue region and echoes from the tissue are sampled along each radial direction which corresponds to different tissue depths. The amplitude of the of the reflected and received acoustic signals is then used to form the images and further process to perform the pressure measurements described herein. While the data may be preprocessed, the raw image data is used herein to avoid any further compression of the data such as via colormap process.

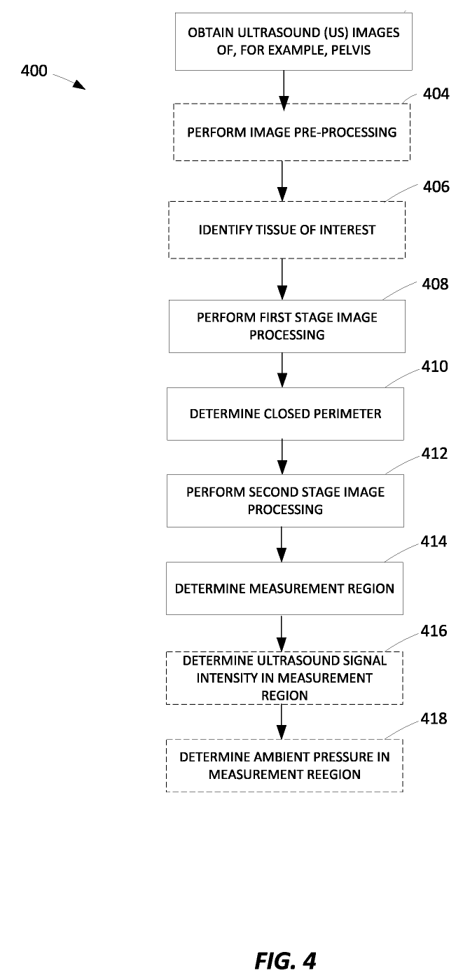

FIG. 4 is a flow diagram of a method 400 for performing pressure measurements in tissues and organs, such as in the bladder. The method 400 may be performed using a system similar to those illustrated in FIGS. 1 and 2, and as such, the method 400 will be described with continued reference to elements of FIGS. 1 and 2 for clarity. Some blocks within FIG. 4 are depicted with dashed lines to indicate that these blocks are optional. The method 400 includes obtaining a plurality of ultrasound images of a body region of a patient at block 402. The body region may include void spaces within the area where the tissue is located, such as the interior of the bladder, or non-tissue features (e.g., medical implants) localized within the area. The images may be obtained using an ultrasound probe, such as the probe 120. The body region includes a contrast agent for improving the signal to noise ratio of the fluid in the tissues or organs, or to increase the ultrasound signal in specific tissues or organs for performing the pressure measurements. For example, the contrast agent may be microbubbles (MBs). In examples, the ultrasound images may be obtained using a wearable device or cart-based ultrasound system as well. For simplicity, the method 400 is described herein with reference to a single ultrasound image referred to as the “first ultrasound image”, but it should be understood that the method 400 may be applied to any number of images for performing pressure measurements over time, and for performing pressure measurements in real-time. For example, the ultrasound probe 120 may obtain a series of images over time to track the pressure of tissues or organs while the tissues move, organ moves, or probe is moved. The ultrasound probe 120 may obtain a first image, and subsequent second, third, etc. images. The ultrasound probe 120 may obtain a video of the body region, and individual frames of the video may be isolated and taken independently as images of the plurality of images. The disclosed methods decouple the images of the tissues and organs from movements and therefore improve the accuracy of pressure measurements across images for tissues or organs that may have changes in position over time.

The ultrasound probe may obtain images at a rate of between 30 and 100 frames per second, 10 to 100 frames per second, 100 to 500 frames per second, 500 to 1000 frames per second, greater than 1000frames per second or at another frequency. The body region may include a bladder, or a portion of a bladder, a fluid, and/or one or more tissues or other organs of the urinary tract. The method 400 is not limited to a particular organ or tissue but is generally applicable to a variety of organs and tissues.

Optionally, the one or more processors 222 may then perform image preprocessing on one or more of the ultrasound images, such as the first ultrasound image, at block 404. The image preprocessing may include performing one or more image transformations, greyscale conversion, histogram equalizations, contrast enhancement, brightness normalization, background subtraction, thresholding, noise removal, etc. For example, the processor may first convert an ultrasound image to greyscale to reduce any color data to a single grey channel to simplify subsequent processing while retaining essential image data and information. A contrast limited adaptive histogram equalization (CLAHE) may then be applied to enhance image contrast. A CLAHE is typically effective in improving the visibility of features in medical imaging by normalizing the brightness and increasing contrast of medical images, such as ultrasound images.

At block 406, the method 400 optionally includes determining one or more areas of interest in the first ultrasound image. The one or more areas of interest may include one or more tissues or organs, fluid or void spaces within tissues or organs, and/or portions of tissues or organs, in the urinary tract. The one or more areas of interest may include, for example, the bladder and fluid within the bladder. In examples, a processor may perform image processing or machine vision processes to determine the areas of interest , or a user may provide a user input to determine the areas of interest and the target elements. To determine the areas of interest , the processor 222 may perform image segmentation including one or more of, without limitation, edge detection, contrast analysis, thresholding, contouring, or one or more morphological operations in addition to other algorithms and image processing techniques.

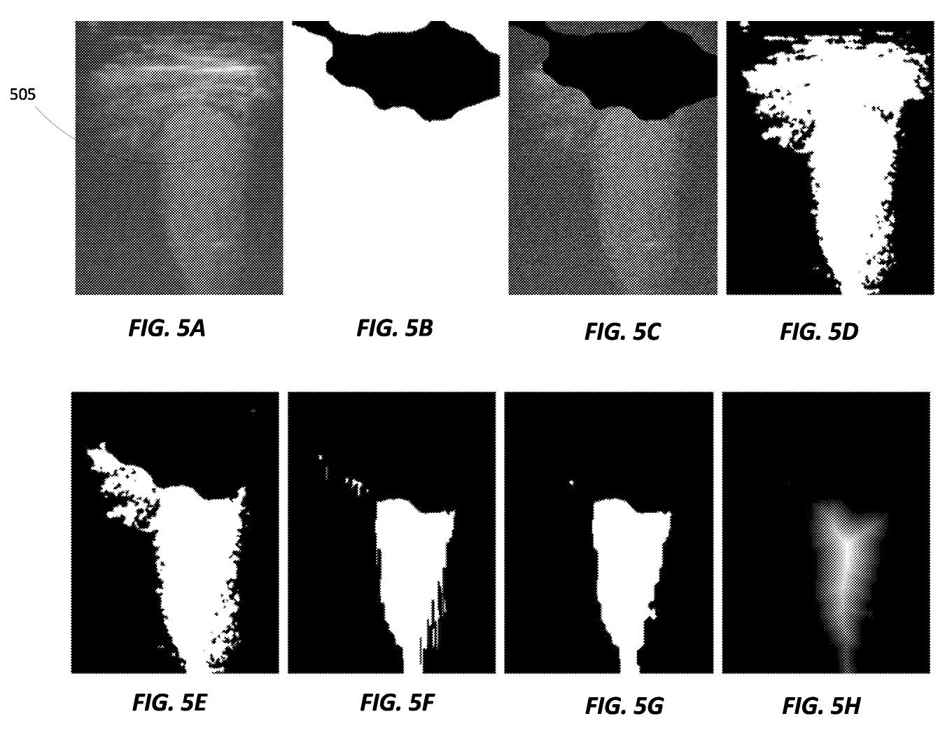

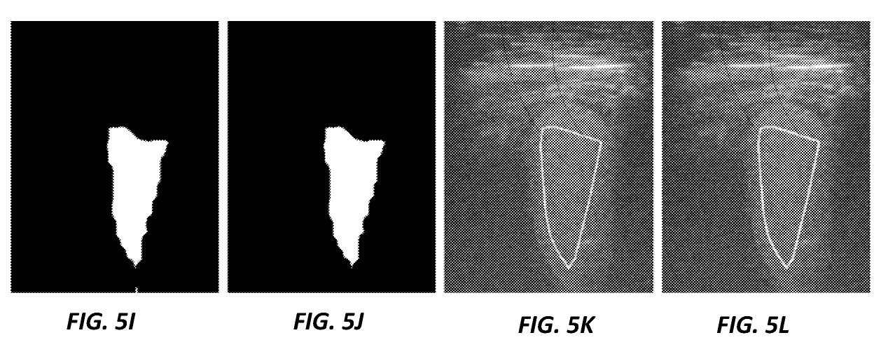

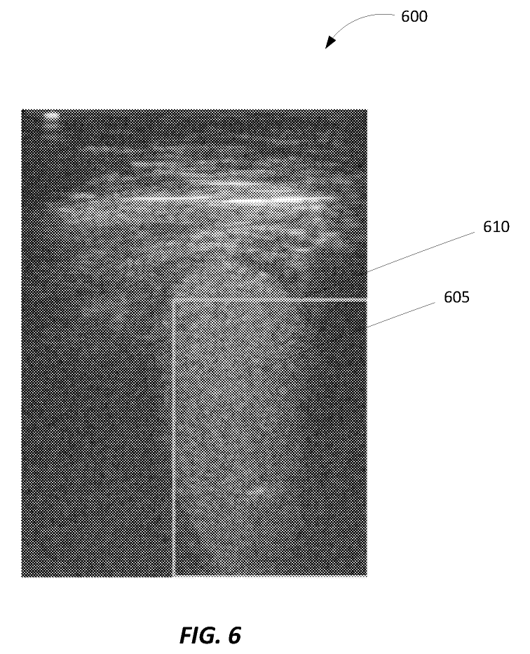

At block 408, the processor 222 performs first stage image processing on the first ultrasound image to determine a closed perimeter, such as a bounding box. The first stage image processing provides a method for delineating between tissue signals that the ultrasound probe has been unable to suppress in a contrast operating mode. FIGS. 5A through 5L present ultrasound images of a bladder 505 and various example image processing steps performed in one implementation of the first stage image processing. FIG. 6 provides the ultrasound image of FIG. 5A, with a closed perimeter 610 indicative of a region of interest that includes the bladder 505 as the area of interest.

While described below in reference to the various image processing techniques illustrated in FIGS. 5A-5M, the first stage image processing may include any number of image processing techniques and additional processing algorithms and methods to identify and determine the closed perimeter 610. For example, the first stage image processing may include one or more of noise reduction (e.g., blurring operations), horizontal tissue layer analysis (e.g., using gradient operators such as Sobel, and edge detection algorithms like Canny) , adaptive thresholding (e.g., Otsu)binarization (e.g., binary thresholding), mask application, morphological operations (e.g., erosion, dilation), smoothing functions for shape refinement, distance transformation for region analysis, small blob removal, convex hull extraction for contour detection, and contour smoothing.

In one implementation, the first stage image processing includes the various techniques and image processing methods illustrated in FIGS. 5A-5M. First, an image blur is performed on the ultrasound (FIG. 5A). The blurring technique is used to reduce high spatial frequency noise so the focus can be on the general structure of organs. Semi-horizontal tissue layers, which have not been suppressed by the scanner in contrast mode, are then identified and a mask layer is derived from the region of the ultrasound image including the semi-horizontal tissue layers. The semi-horizontal tissue layers are determined by gradient operators such as Sobel algorithm and edge detection techniques such as Canny algorithm. FIG. 5B provides an image of a mask layer over the regions of the ultrasound image that include the semi-horizontal tissue layers, that are not to be considered in the pressure measurement. The mask layer shown in FIG. 5B is then used to remove the portion of the ultrasound image that contains the semi-horizontal tissue layers. FIG. 5C provides an image of the ultrasound image with the portion of the image with the semi-horizontal tissue removed according to the determined mask from FIG. 5B.

An Otsu thresholding technique is applied to the ultrasound image of FIG. 5A to identify pixels as foreground pixels having image content pertaining to contrast agent in an object (e.g., organ or tissues) and background pixels having no contrast agent for performing pressure measurements. The Otsu thresholding identifies the different types of pixels by implementing a minimization of intensity variances across pixels in regions and groups. FIG. 5D provides a resultant image mask of determined foreground pixels (white/lighter portions of the image), and the background pixels (black/darker portions of the image), using the Otsu thresholding. The resultant Otsu mask is combined with the determined mask layer of the semi-horizontal tissues to remove the background pixels, and the semi-horizontal tissues from the image data. FIG. 5E provides an image of the resultant mask of the foreground pixels (white/bright pixels) to consider further for performing additional image processing and performing a pressure measurement.

An erosion filter is then applied to the foreground pixels (FIG. 5F). The erosion filter is performed to remove small, disconnected foreground pixel regions and to refine the boundaries of the detected regions. This helps in eliminating noise and small artifacts that may falsely be identified as part of the object of interest. The operation improves the specificity of the segmentation by shrinking the foreground regions and isolating more defined shapes for subsequent processing. A smoothing filter is then applied to reduce the noise in the image and remove small potential errors and smooth edges of the resultant foreground pixels (FIG. 5G).

A distance transform is then applied to the resultant foreground pixels (FIG. 5H). The distance transform identifies a centroid, central region, axis, or axis for the resultant foreground pixels and then applies a gradient dependent on how far a given pixel is from the central axis, point, or region. The resultant image of FIG. 5H shows the determined central region of the foreground pixels (i.e., the bright central region), and pixel values are decreased (i.e., decreased brightness) at increased distances from the centroid, or identified central axis or region. The distance transform is performed to quantify the spatial relationship of each pixel in the foreground to the central region, centroid, or central axis of the shape. This helps in identifying the geometric properties of the detected region, such as symmetry and elongation. By assigning a gradient-based value to pixels depending on their distance from the center, it effectively enhances the ability to identify consistent shapes and reduces noise by emphasizing structured spatial relationships. This step contributes to increasing the signal-to-noise ratio (SNR) by accentuating the primary structure while suppressing irrelevant details.

An additional binary threshold filter is then applied to the distance threshold image of FIG. 5H to identify pixels of interest for performing the pressure measurement FIG. 5I is an image with the binary thresholding applied to the distance threshold image of FIG. 5H. The binary threshold determines background pixels (dark pixels of FIG. 5I) as pixels having a pixel value below a certain pixel brightness value and determines pixels of interest (bright pixels of FIG. 5I) as pixels having a pixel value above a certain pixel brightness value in the distance transformed image.

An additional filter, such as a morphological opening and/or a connected-component analysis, is then applied to remove small groups of pixels outside of the main pixels of interest. FIG. 5J presents an image where a small group of white pixels has been remove from the center bottom portion of the image shown in FIG. 5I. Small groups of pixels of interest, outside of the main grouping of pixels of interest, may simply be determined as pixels of interest due to noisy images or readings, or due to the imaging of tissues of other organs that were captured by a probe and that should not be considered when performing a pressure measurement on an organ such as a bladder. Removal of the additional small regions of pixels of interest reduces the potential noise in the readings and may improve the accuracy of pressure measurements of specific organs or body regions.

Performing first stage image processing may then further include performing a standard computational geometry algorithm such as Graham’s scan or Quickhull to determine a convex hull or convex outline of the resultant mask or pixels of interest. FIG. 5K provides an image of an outline of the convex hull overlaid on the original ultrasound image of the tissue of regions including the bladder 510. The convex hull generally includes the pixels of interest as presented in FIG. 5J, but further reduces curves in the various edges of the mask. The convex hull simplifies the shape of the detected region by reducing local irregularities and small indentations, creating a smoother and more generalized boundary. A final smoothing function is performed on the edges of the resultant convex hull 510 to further reduce any small variations, bumps, or errors in the outline of the convex hull 510. FIG. 5L provides an image of the final convex hull 510 overlaid on the original ultrasound image of the bladder 505.

At block 410 of the method 400, one or more processors determine a closed perimeter from the final convex hull 510. In this example, the closed perimeter is a bounding box. The closed perimeter is indicative of a region of interest of the ultrasound image that includes one or more tissues or organs, or portions of tissues or organs, of interest for performing the pressure measurement. FIG. 6 shows the first ultrasound image used in the first stage processing of FIGS. 5A-5M, with the bladder 605 and the determined closed perimeter 610. In the example provided, the closed perimeter 610 is determined by coordinates of the resultant mask of FIG. 5L. The top left corner of the closed perimeter of FIG. 6 is taken as the top left point of the resultant mask of FIG. 5L. In this example, the bounding box is then defined as a rectangular box extending down to the bottom of the ultrasound image, and to the right edge of the ultrasound image or to the right most and bottom most point of the contour. In the example of the bladder 605 of FIG. 6, a large portion of the bladder is included in the closed perimeter by extending the bottom and right sides of the closed perimeter 610 to the edges of the ultrasound image. While illustrated as a rectangle, the closed perimeter 605 may be another polygon, a circle, an ellipse, an asymmetric shape, or another perimeter that is determined from the first image processing to identify a region of interest, and areas of interest for analyzing ultrasound signals and performing a pressure measurement.

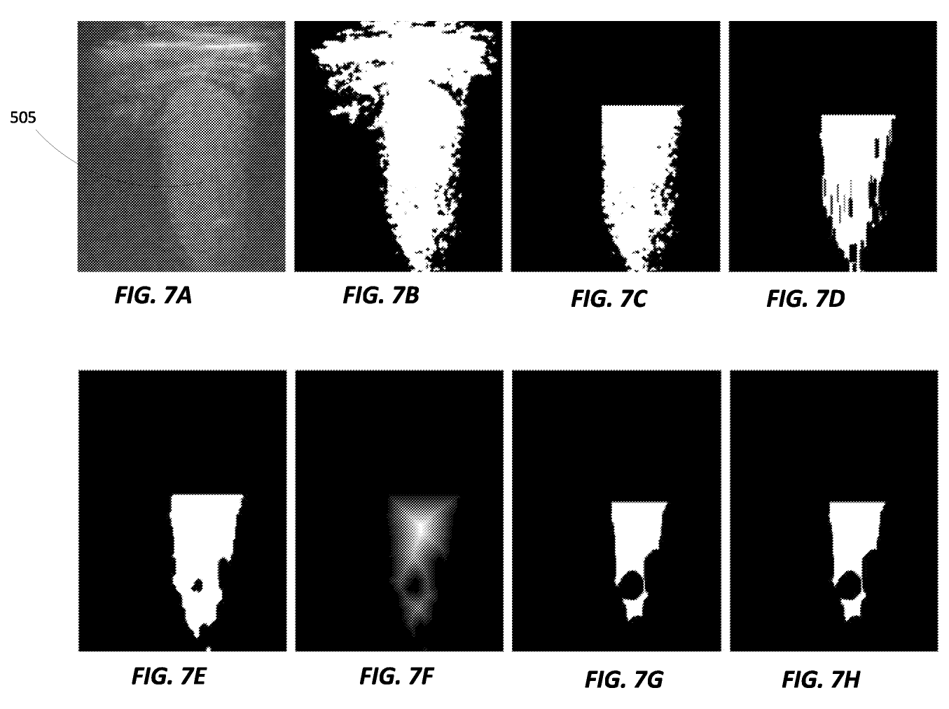

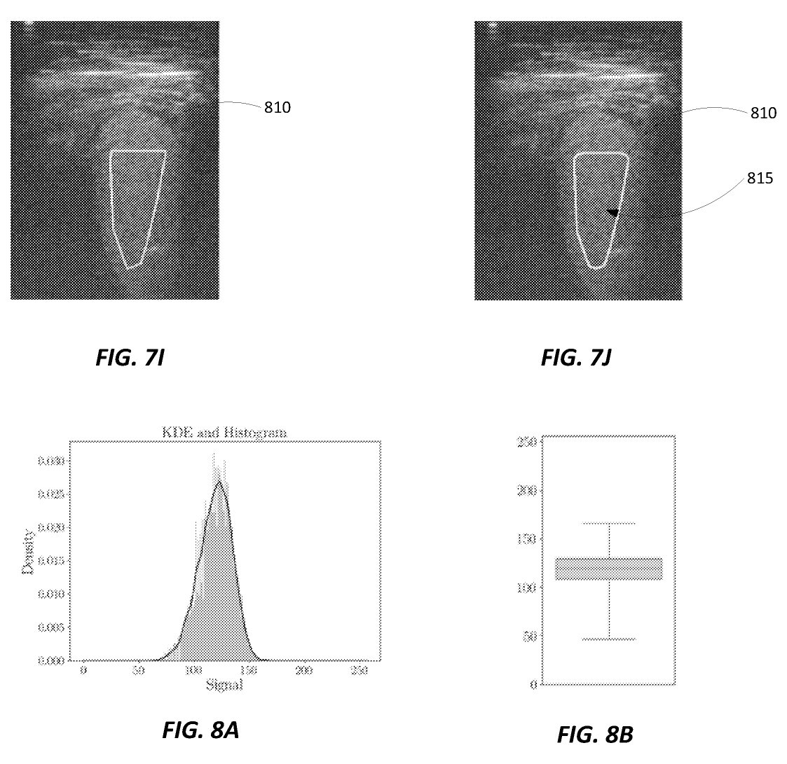

After the closed perimeter 610 is determined, the method 400 further includes performing second stage image processing on the first ultrasound image, at block 412. One or more processes of the second stage image processing are performed on the region of interest of the ultrasound image as indicated by the closed perimeter 610. Limiting the image processing to pixels of the region of interest allows for focused image processing to reduce image noise and improve the accuracy of resultant pressure measurements from the ultrasound images. Omitting unnecessary regions of the image allows for a more focused analysis and approach for performing the pressure measurement. FIGS. 7A through 7L present ultrasound images of the bladder 505 and various example image processing steps performed in one implementation of the second stage image processing. The second stage image processing further determines a measurement region of an ultrasound image to further analyze the ultrasound data and perform a pressure measurement.

While described below in reference to the various image processing techniques illustrated in FIGS. 7A-7L, the second stage image processing may include any number of image processing techniques and additional processing algorithms and methods to determine a measurement region of an ultrasound image to perform a pressure measurement. The second stage image processing may utilize one or more of the same imaging processing techniques, algorithms, or methods, but may use stricter parameters than those used in the first stage image processing. For example, the second stage image processing may include one or more of an image blur, semi-horizontal tissue layer analysis, an Otsu thresholding technique, a binary thresholding, application of a mask, an erosion, a smoothing function, a distance transform, a contour function, a spline, a thresholding technique, or another image or data processing operation.

In the provided example, the second stage image processing first applies a blur to the first ultrasound image (FIG. 7A), and then performs an Otsu thresholding operation to generate a binary mask of foreground (bright/white pixels), and background (dark/black pixels), as shown in FIG. 7B. The closed perimeter 610 determined from the first stage image processing is then applied and pixels outside of the closed perimeter 610 (i.e., outside of the region of interest) are omitted and not considered for further processing (FIG. 7C). An erosion is applied to the resultant foreground pixels, also referred to as pixels of interest (FIG. 7D), and a smoothing function is applied to reduce the noise and textures of the edges of the resultant foreground pixels (FIG. 7E). A distance transform is performed (FIG. 7F) and a binary threshold filter is applied to limit the set of pixels of interest to pixels within a certain distance from a centroid, central axis, or central region of the foreground pixels (FIG. 7G). Small regions of foreground pixels are then identified and removed to prevent noise from strong ultrasound reflections for undesired tissues or outlier noise from being taken into account during the pressure measurement analysis (FIG. 7H). The small regions of foreground pixels may be determined as a set of adjacent pixels under a specific limit, or as any groups of pixels not directly adjacent to the largest set of foreground pixels in a given image.

A convex hull is then identified using the final set of pixels of interest from FIG. 7H. FIG. 7I provides an image of a convex hull 810 bounding a portion of the bladder 605 for performing a pressure measurement. One or more processors then performs an edge smoothing function to further reduce any noise or sharp transitions along the edges of the convex hull 810 (FIG. 7J).

At block 414 a measurement region is determined in the first ultrasound image. The measurement region is indicative of a set of pixels of the contrast-enhanced ultrasound image that are to be further analyzed for performing a pressure measurement. In the current example, the convex hull 810 of FIG. 7J outlines a measurement region 815 of pixels for performing a pressure measurement. The measurement region 815 is identified as a sub-region of the closed perimeter 610, and further by second stage image processing. One or more processors further analyze the ultrasound signal in the measurement region to perform a pressure measurement.

Optionally, at block 416 one or more processors determines the ultrasound contrast signal intensity in the measurement region 815 of the first ultrasound image. Determining an ultrasound contrast signal intensity in the measurement region of the first ultrasound image may include extracting a contrast signal. Determining the ultrasound contrast signal intensity may further include identifying one or more subharmonic ultrasound signal intensities from the ultrasound contrast signal intensity.

Optionally, at block 418, the method 400 further includes one or more processors determining, from the subharmonic signal intensity, a pressure in the measurement region 815. To determine the pressure the processor(s) may first determine a distribution of ultrasound contrast signal intensity in the measurement region 815 and its mean, and may then determine the ambient pressure by SHAPE technique. For example, for a given patient, organ, or tissue, a processor may use a lookup table or predetermined correlation relation to convert the ultrasound signal or determined conversion factor.

FIG. 8A provides a histogram of a pixel distribution of ultrasound contrast signal intensity for the ultrasound image, and specifically for the measurement region 815 of FIG. 7J. FIG. 8B is a box plot of the ultrasound intensity of the measurement region 815 of FIG. 7J. The box plot shows a mean ultrasound intensity of 112, and associate upper and lower quartiles, and maximum and minimum values indicated by the whiskers. The histogram and box plot of FIGS. 8A and 8B are one example of metrics and analysis that may be used for evaluating the ultrasound contrast signal intensity, and further, for performing a pressure measurement. Other statistical methods and analysis may be used.

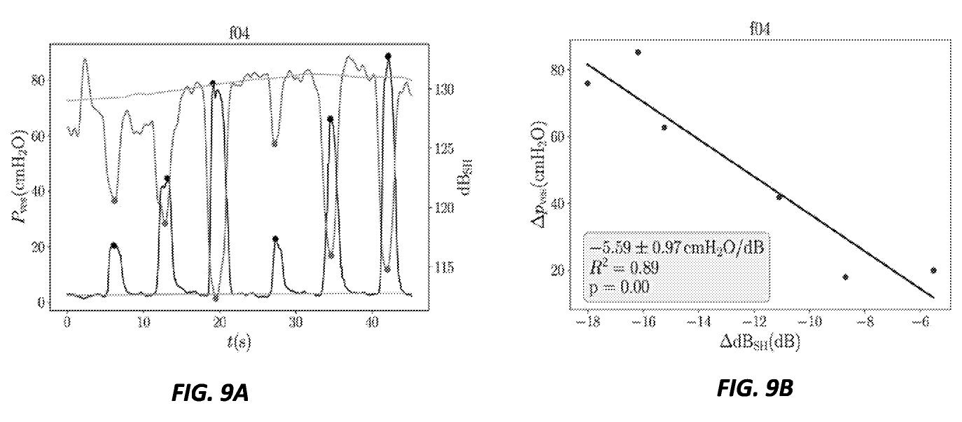

FIG. 9A illustrates an example of correlation between bladder pressure Pᵥₑₛ and microbubble subharmonic signal amplitude A_{sub} for a filling phase of urinary activity obtained with an ultrasound contrast agent. Both the phantom pressures (left axis) and microbubble subharmonic signal amplitudes (right axis) are plotted, demonstrating a typical inverse relationship where each pressure spike is accompanied by a corresponding drop in subharmonic signal. The peaks are shown with circles for pressure, and the valleys are shown with circles for sub-harmonic signal. The signal baselines are depicted with solid lines at the top and bottom of the graph. FIG. 9B illustrates graphically the linear relationship between change in pressure and change in subharmonic signal. The slope of linear fit is conversion factor that can be used to convert change in subharmonic signal to change in pressure.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the target matter herein.

Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed:

1. A method for performing automated segmentation of contrast enhanced ultrasound images across the urinary tract, the method comprising:
obtaining, by an ultrasound sensor, a first ultrasound image of a body region of a patient, the body region including one or more tissues or organs of the urinary tract;
identifying an area of interest in the first ultrasound image;
performing first stage image processing on the first ultrasound image and determining a closed perimeter in the first ultrasound image from the first stage image processing, the closed perimeter indicative of a region of interest of the first ultrasound image that includes a portion of the area of interest;
performing second stage image processing on the first ultrasound image, including limiting at least part of the second stage image processing to the region of interest indicated by the closed perimeter; and
determining a measurement region in the first ultrasound image from the second stage image processing and the region of interest indicated by the closed perimeter.

2. The method of claim 1 further comprising determining an ultrasound contrast signal intensity in the measurement region of the first ultrasound image.

3. The method of claim 2, wherein determining the ultrasound contrast signal intensity comprises identifying one or more subharmonic signal intensities.

4. The method of claim 3 further comprising determining an ambient pressure in the measurement region from the ultrasound contrast signal intensity.

5. The method of claim 1, wherein the first stage image processing includes one or more of noise reduction, horizontal tissue layer analysis, adaptive thresholding, binarization, mask application, an erosion, a dilation, a smoothing function for shape refinement, a distance transformation for region analysis, small blob removal, convex hull extraction for contour detection , and contour smoothing.

6. The method of claim 1, wherein the second stage image processing includes applying, for the region of interest indicated by the closed perimeter, one or more of noise reduction, horizontal tissue layer analysis, adaptive thresholding, binarization, mask application, an erosion, a dilation, a smoothing function for shape refinement, a distance transformation for region analysis, small blob removal, convex hull extraction for contour detection , and contour smoothing.

7. The method of claim 4, wherein determining the ambient pressure from the ultrasound contrast signal intensity comprises determining a distribution of ultrasound signal within the measurement region, and correlating ambient pressure in the measurement region with the distribution of ultrasound signal.

8. The method of claim 7, wherein determining the distribution of ultrasound signal comprises generating a histogram of ultrasound signal in the measurement region.

9. A system for automated segmentation of contrast enhanced ultrasound images from across the urinary tract, the system comprising:
an ultrasound sensor;
a processor configured to execute machine readable instructions; and
a non-transitory computer-readable memory having machine-readable instructions stored thereon, that when executed by the processor, cause the system to:
obtain, by the ultrasound sensor, a first ultrasound image of a body region of a patient, the body region including one or more tissues or organs of the urinary tract;
identify an area of interest in the first ultrasound image;
perform first stage image processing on the first ultrasound image and determine a closed perimeter in the first ultrasound image from the first stage image processing, the closed perimeter indicative of a region of interest of the first ultrasound image that includes a portion of the area of interest ;
perform second stage image processing on the first ultrasound image, including limiting at least part of the second stage image processing to the region of interest indicated by the closed perimeter; and
determine a measurement region in the first ultrasound image from the second stage image processing and the region of interest indicated by the closed perimeter.

10. The system of claim 9, wherein the machine-readable instructions, when executed by the processor, cause the system to determine an ultrasound contrast signal intensity in the measurement region of the first ultrasound image.

11. The system of claim 10, wherein determining the ultrasound contrast signal intensity comprises identifying one or more subharmonic signal intensities.

12. The system of claim 11, wherein the machine-readable instructions, when executed by the processor, cause the system to determine an ambient pressure in the measurement region from the ultrasound contrast signal intensity.

13. The system of claim 9, wherein the first stage image processing includes
one or more of noise reduction, horizontal tissue layer analysis, adaptive thresholding, binarization, mask application, an erosion, a dilation, a smoothing function for shape refinement, a distance transformation for region analysis, small blob removal, convex hull extraction for contour detection , and contour smoothing.

14. The system of claim 9, wherein the second stage image processing includes
one or more of noise reduction, horizontal tissue layer analysis, adaptive thresholding, binarization, mask application, an erosion, a dilation, a smoothing function for shape refinement, a distance transformation for region analysis, small blob removal, convex hull extraction for contour detection , and contour smoothing.

15. The system of claim 12, wherein to determine the ambient pressure from the ultrasound contrast signal intensity, the machine-readable instructions, when executed by a processor, further cause the system to determine a distribution of ultrasound signal within the measurement region, and correlate ambient pressure in the measurement region with the distribution of ultrasound signal.

16. The system of claim 15, wherein to determine the distribution of ultrasound signal the machine-readable instructions, when executed by a processor, cause the system to generate a histogram of ultrasound signal in the measurement region.

17. One or more non-transitory computer-readable media storing computer executable instructions that, when executed via one or more processors, cause one or more systems to:
obtain a first ultrasound image of a body region of a patient, the body region including one or more tissues or organs of the urinary tract;
identify an area of interest in the first ultrasound image;
perform first stage image processing on the first ultrasound image and determine a closed perimeter in the first ultrasound image from the first stage image processing, the closed perimeter indicative of a region of interest of the first ultrasound image that includes a portion of the area of interest ;
perform second stage image processing on the first ultrasound image, including limiting at least part of the second stage image processing to the region of interest indicated by the closed perimeter; and
determine a measurement region in the first ultrasound image from the second stage image processing and the region of interest indicated by the closed perimeter.

18. The computer-readable media of claim 17, the computer-readable media storing computer executable instructions that, when executed via one or more processors, cause one or more systems to determine an ultrasound contrast signal intensity in the measurement region of the first ultrasound image.

19. The computer-readable media of claim 18, the computer-readable media storing computer executable instructions that, when executed via one or more processors, cause one or more systems to determine an ambient pressure in the measurement region from the ultrasound contrast signal intensity.

20. The computer-readable media of claim 18, wherein to determine the ultrasound contrast signal intensity, the computer executable instructions, when executed via one or more processors, cause the one or more systems to:
identify one or more subharmonic signal intensities;
determine a distribution of the one or more subharmonic signal intensities within the measurement region; and
correlate ambient pressure in the measurement region with the distribution of subharmonic signal intensities.
